(19) United States
(12) Patent Application Publication
(10) Pub. No.: US 2017/0128356 A1
(43) Pub. Date: May 11, 2017

(54) SKINCARE STIMULANT HAVING A PLATELET DRY POWDER

(71) Applicant: CENTRAL MEDICAL TECHNOLOGIES INC., Taipei (TW)

(72) Inventors: Chih I LIN, Taipei (TW); Han-Lei WAN, Taipei (TW)

(73) Assignee: CENTRAL MEDICAL TECHNOLOGIES INC., Taipei (TW)

(21) Appl. No.: 15/406,266

(22) Filed: Jan. 13, 2017

Related U.S. Application Data
(63) Continuation-in-part of application No. 13/288,055, filed on Nov. 3, 2011, now abandoned.

(30) Foreign Application Priority Data
Nov. 9, 2010 (TW) 99138587

(51) Int. Cl. A61K 8/98; A61H 23/00; A61M 35/00; A61M 37/00; A61Q 19/08; A61K 35/19

(52) U.S. Cl. CPC A61K 8/983; A61Q 19/08; A61K 35/19; A61M 35/00; A61M 37/0015; A61H 23/006; A61K 2035/124

(57) ABSTRACT

A skincare stimulant having an effective dose of platelets and pharmaceutically acceptable solvents and/or excipients, where the effective dose refers to the presence of at least 1000 platelets in every milliliter of skincare stimulant. A method for treating a skin condition selected wrinkles, amyloidosis and keratosis pilaris includes spraying or spreading the skincare stimulant having over a body area, where the skincare stimulant is a solution having a concentration of 1000-10000 platelets per milliliter of the solution, wherein a source of the platelets is a platelet dry powder having less than 30% of plasma protein, based on the weight of the platelet dry powder.

### CROSS REFERENCE TO RELATED APPLICATIONS

This patent application is a continuation-in-part application of U.S. patent application Ser. No. 13/288,055, filed Nov. 3, 2011, now abandoned, which claims the priority benefit of Taiwan Patent Application Number 99138587, filed Nov. 9, 2010. The disclosures of these prior patent applications are incorporated herein by reference.

### FIELD OF THE INVENTION

The present invention relates to a skincare stimulant and usage thereof, and more particularly to a skincare stimulant using compositions of platelets for ameliorating skin aging and usage thereof

### BACKGROUND OF THE INVENTION

The skincare industry has developed important techniques to resolve problems like wrinkles, loose skins, pock marks, enlarged pores, dark spots, and hyperpigmentation, as can be referred to in the published Taiwan patent 201018492, a skincare composition based on peptides that inhibits the reactiveness of the reactive carbonyl species (RCS) was disclosed, which is applicable to skin, mucous membrane, scalp, and/or hair treatments, and/or skincare; in the published Taiwan patent 200812633, a technique employing whey protein micelles as a grinding agent was disclosed. In the announced Taiwan patents 1281405 and 1260231, and the published Taiwan patents 200735893 and 200505492, formulations of skincare stimulant using small-molecule chemicals as the active ingredients were disclosed, yet a skincare stimulant using a platelet dry powder as the active ingredient has not been developed so far. In the US patents, the only patent that had disclosed a skincare stimulant using platelets-related substances as the active ingredient is the published patent 20070253940, and the patent disclosed: a skincare stimulant using the platelet-rich plasma as the active ingredient. The present invention is the first to have disclosed: a skincare stimulant utilizing a platelet dry powder as the active ingredient, which achieves actual effects such as anti-wrinkling, removing pigments, reducing skin lines, increasing skin elasticity/tightening skin, and also stimulating and renewing dermal cells to rebuild skin tissues, and allays amyloidosis and keratosis pilaris. In addition, the use of this skincare stimulant achieves stronger, more comprehensive, and longer-lasting effects than that of the traditional methods.

### SUMMARY OF THE INVENTION

The invention provides a skincare stimulant using platelets as the active ingredient.

The invention provides platelets for anti-wrinkling effect.

The invention provides platelets for removing pigmentation.

The invention provides platelets for reducing skin lines and increasing skin elasticity/tightening skin.

The invention provides platelets for stimulating the renewal of dermal cells.

The invention provides platelets for rebuilding skin tissues.

The invention provides platelets for ameliorating amyloidosis.

The invention provides platelets for ameliorating keratosis pilaris.

The skincare stimulant of the invention includes an effective dose of platelets and pharmaceutically acceptable solvents and/or excipients, wherein the effective dose refers to the presence of at least 1000 platelets in every milliliter of skincare stimulant.

The source of said platelets of the skincare stimulant of the present invention is a platelet dry powder. Said platelet dry powder is prepared from blood or blood preparations like platelet-rich plasma (abbreviated as PRP hereafter) by using specific methods, and comprises intact platelets and less than 30% of plasma protein to avoid possible allergic reactions, based on the weight of the platelet dry powder. The methods for preparing the platelet dry powder may be any of the previously known methods for preparing platelet dry powders, as can be referred to in the announced Taiwan patents 1300806 and 1270375; the published Taiwan patents 201004659 and 200526680, and the announced US patents 7659052, 7202020, 7169606, 6060233, 5736313, and 5589462. Moreover, the preparation of the platelet dry powder described in this invention is not limited to the methods disclosed in the aforesaid cited references. Said platelet dry powder may comprise low amounts of anticoagulants and protectants from using different methods, such as the platelet dry powder prepared according to the method disclosed in the announced Taiwan patent TW-I270375, which includes low amounts of the anticoagulant acid citrate dextrose, cryoprecipitates, and thrombin, but since said components do not actually affect the performance of the platelet dry powder, it is not necessary to eliminate them therefrom.

Said skincare stimulant may be any of the previously known forms of drugs, such as solutions, suspensions, ointments, powders, and pills; is preferably to be in the form of solutions, ointments, or transdermal patches, and is more preferably to be in the form of sprays or ointments. Moreover, the powders and pills may be prepared into other forms suitable for use in actual applications.

Said pharmaceutically acceptable solvents refer to any solvents that can be ingested or applied externally by humans or animals, such as alcohol-water co-solvents, water, and saline, and is preferably water or saline. It is also necessary to ensure the amount of solvents added could maintain the effective dose of platelets.

The pharmaceutically acceptable excipients are the previously known excipients, and applications thereof are determined according to the forms of drugs.

Said effective dose of the platelets refers to the presence of at least 1000 platelets in every milliliter of the skincare stimulant; is preferably to be the presence of at least 3000 platelets therein, and is more preferably to be the presence of at least 5000 platelets therein. Generally, the more the platelets, the more significant the resulted effects. For instance, a skincare stimulant having 20000 platelets in every milliliter thereof has been shown to achieve more significant effects than a skincare stimulant having 5000 platelets in every milliliter thereof

Said skincare stimulant may be added with any effective ingredients that convey positive effects thereto (such as anti-inflammatory ingredients, analgesic ingredients, nutritious ingredients, and/or ingredients promoting absorption), or any ingredients that do not have negative effects or side effects (such as spices).

Said platelet dry powder may be heterologous, homologous, or autologous platelet dry powders. Considering the users' possible concerns, the platelet dry powder is preferably homologous or autologous platelet dry powders, and is more preferably autologous platelet dry powders.

Generally speaking, the platelet-derivatives growth factors (PDGF) of platelets in blood are approximately 40-200 pg/mL, as can be referred to in Vogt., et al., Determination of endogenous growth factors in human wound healing. Wound Repair Regeneration, 2004, 12(4): p. 485-492. The effectiveness of the skincare stimulant of the invention may be related to the long-term effectiveness of PDGF of the platelet dry powder, but the relationship thereof still requires further investigation.

A method for treating a skin condition selected from the group consisting of wrinkles, amyloidosis and keratosis pilaris, according to the invention, comprising:

a step for identifying a body area having a condition selected from the group consisting of wrinkles, amyloidosis and keratosis pilaris; and

a step for spraying or spreading a skincare stimulant having platelets over said body area, wherein the skincare stimulant is a solution having a concentration of 1000-10000 platelets per milliliter of the solution;

wherein a source of the platelets is a platelet dry powder having less than 30% of plasma protein, based on the weight of the platelet dry powder.

Preferably, the method further comprises a step for cleaning the body area before said skincare stimulant being sprayed or spread over the body area.

Said cleaning step refers to steps like rinsing and disinfecting to induce the body area awaiting the stimulation of skincare into a condition suitable for further treatments. Generally, the need of rinsing body area awaiting treatments depends on the cleanness of the body area, and the body area are preferably rinsed in advance or immediately before treatments. Though disinfecting is not absolutely required, it is preferable to have the body area disinfected thoroughly.

Said spraying or spreading step refers to spraying or spreading the skincare stimulant having an effective dose of platelet dry powder over said cleaned body area according to the form of the skincare stimulant. For example, if the skincare stimulant is a solution, it can be evenly sprayed on the body area awaiting the stimulation of skincare, and if the skincare stimulant is an ointment, it can be evenly spread on the body area awaiting the stimulation of skincare.

The details of said skincare stimulant are as described above. Said method for stimulating skincare is suitable to be used on full body skin, especially on the exposed skin such as facial and neck skin.

To enhance the effects of skincare stimulation, a further absorption-enhancing step may be carried out once or multiple times, and said step may be added before, during, or after the spraying or spreading step. Said absorption-enhancing step may be applied in any ways that facilitates the absorption of a skincare stimulant, such as the methods of iontophoresis, laser treament, electromagnetic wave guiding, micro-needle guiding, physical stimulation, and tap massaging that are previously known in the skincare industry. Before the spraying or spreading step, said absorption-enhancing step is preferably carried out by using laser treatment, micro-needle guiding, and physical stimulation (so as to create micro wounds in the stratum corneum to facilitate absorption). The spraying or spreading step may also be carried out by using iontophoresis or electromagnetic wave guiding to facilitate absorption. After the spraying or spreading step, the absorption-enhancing step is preferably carried out by using electromagnetic wave guiding or tap massaging (so as to induce blood circulation to local skin and facilitate absorption).

### BRIEF DESCRIPTION OF DRAWINGS

FIGS. 1a and 1b are photographs showing the patient in Embodiment 1 before and after the treatments.

FIGS. 2a and 2b are photographs showing the patient in Embodiment 2 before and after the treatments.

FIGS. 3a and 3b are photographs showing the patient in Embodiment 3 before and after the treatments.

FIGS. 4a and 4b are photographs showing the patient in Embodiment 4 before and after the treatments.

FIGS. 5a and 5b are photographs showing the patient in Embodiment 5 before and after the treatments.

FIGS. 6a and 6b are photographs showing the patient in Embodiment 6 before and after the treatments.

FIGS. 7a and 7b are photographs showing the patient in Embodiment 7 before and after the treatments.

FIGS. 8a and 8b are photographs showing the patient in Embodiment 8 before and after the treatments.

FIGS. 9a and 9b are photographs showing the patient in Embodiment 9 before and after the treatments.

FIGS. 10a and 10b are photographs showing the patient in Embodiment 10 before and after the treatments.

FIGS. 11a and 11b are photographs showing the patient in Embodiment 11 before and after the treatments.

FIGS. 12a and 12b are photographs showing the patient in Embodiment 12 before and after the treatments.

### DETAILED DESCRIPTION OF PREFERRED EMBODIMENTS

The preferred embodiments are described hereafter in order to further elucidate the techniques of the invention:

### Testing Example:

The method disclosed in the announced Taiwan patent TW-I270375 was used to prepare a platelet dry powder. 1.0 g of platelet dry powder was obtained and added to saline to make up 5.0 mL of solution, then the PDGF titer of the solution was analyzed by using a spectrophotometer (U.S. Bio-Tek Instruments, Inc., Model μ-Quant) immediately after preparation (0 hour), after 1 week (168 hours), after 2 weeks (336 hours), after 3 weeks (504 hours), and after 4 weeks (672 hours).

Results from the analyses are shown in Table 1.

TABLE 1
Changes of PDGF titer in the solution prepared
from the platelet dry powder.

| Weeks | 0 | 1 | 2 | 3 | 4 | 5 | 6 |
|---|---|---|---|---|---|---|---|
| PDGF | 2930 | 2795 | 3012 | 2854 | 2822 | 2776 | 2785 |
| (pg/mL) | | | | | | | |

The experimental figures show: The PDGF titer of the platelet dry powder is very stable.

### Embodiment 1

The method disclosed in the announced patent TW-I270375 was used to prepare an autologous platelet dry powder from autologous blood in advance. The platelet dry powder was added to water obtained by reverse osmosis (RO) to prepare a solution having 5000 platelets/mL therein, then the solution was held in a spray bottle. The number of platelets in the solution was measured by using a Hematology Analyzer (Manufacturer: Sysmex, Model: KX-21).

A patient was allowed to undergo a treatment for skincare stimulation once a week, which involved firstly disinfecting a body area awaiting the skincare stimulation, and then carrying out laser treatment on the disinfected body area, followed by evenly spraying the skincare stimulant (solution) on the disinfected body area. In addition, between every two treatments, the body area awaiting the skincare stimulation was evenly sprayed with the skincare stimulant after washing face everyday.

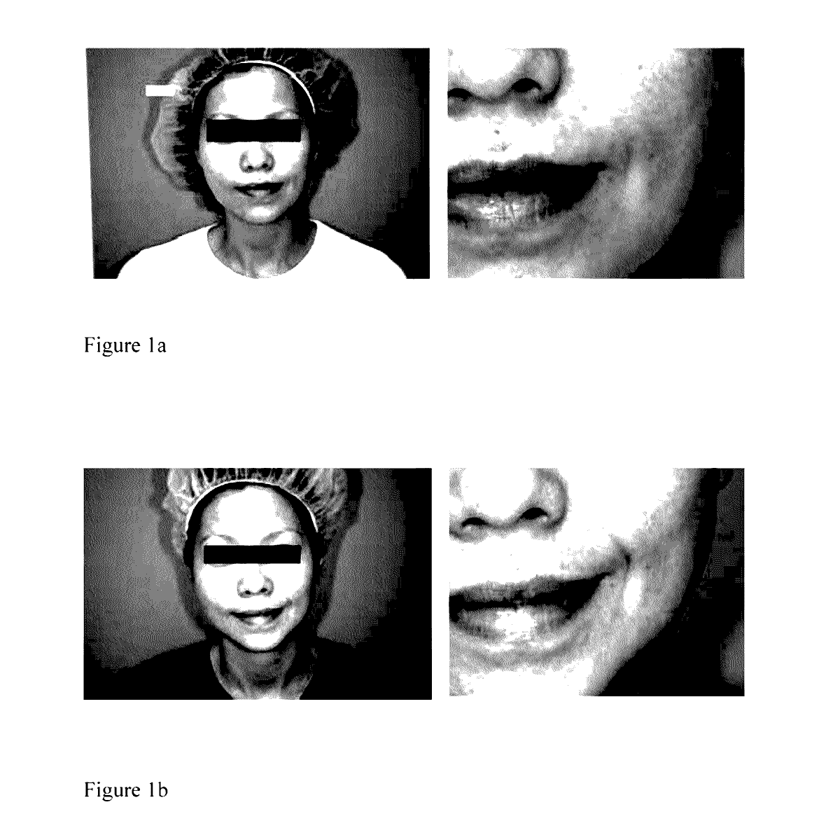

The FIGS. 1a and 1b show the conditions of the body area awaiting the skincare stimulation before the treatments and 4 weeks after the treatments, wherein the photographs on the right are the partially magnified pictures of the photographs on the left. The results showed: the body area awaiting the skincare stimulation showed significantly increased skin elasticity and firmness; the pores became smaller and the skin became whiter and clearer.

### Embodiment 2

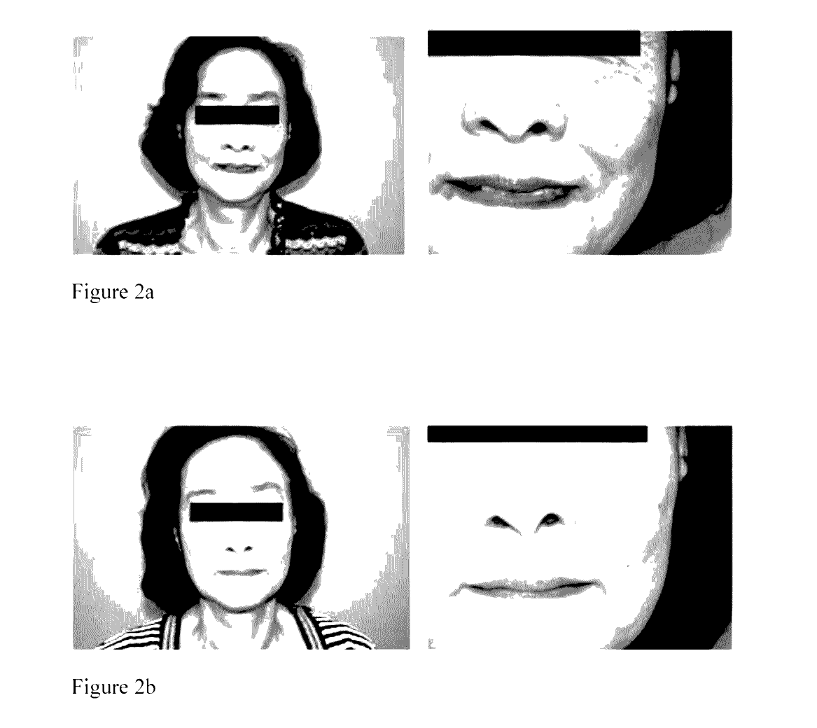

Similar to Embodiment 1, the FIGS. 2a and 2b show the conditions of the body area awaiting the skincare stimulation before the treatments and 7 weeks after the treatments, wherein the photographs on the right are the partially magnified pictures of the photographs on the left. The results indicated: the body area awaiting the skincare stimulation had wrinkles that either vanished or became less apparent, showed significantly increased skin elasticity and firmness; the pores became smaller and the skin became whiter and clearer.

### Embodiment 3

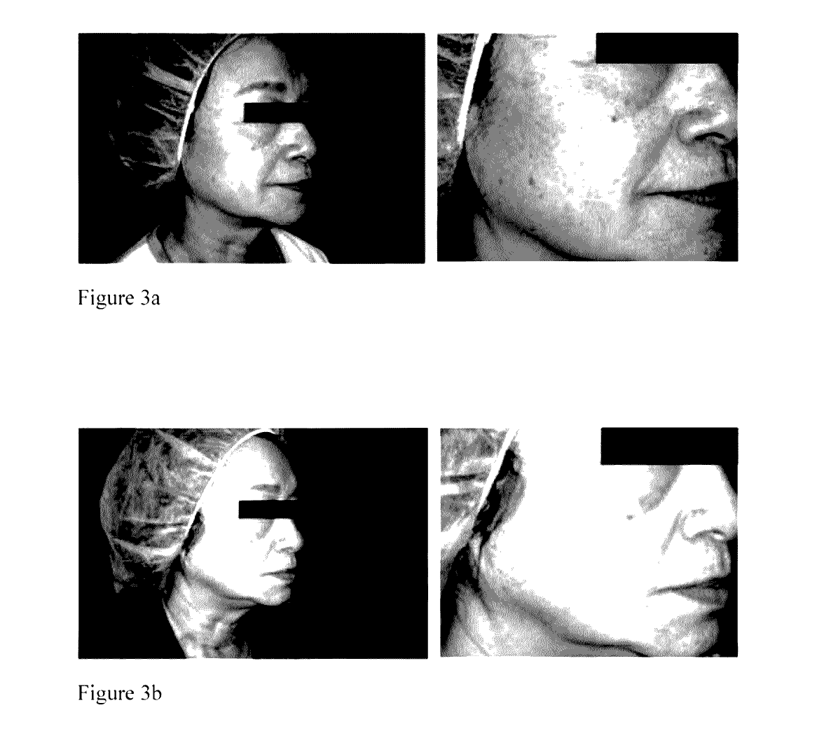

Similar to Embodiment 1, but the platelet dry powder was a homologous platelet dry powder prepared from the blood in a blood bank, and the concentration of the skincare stimulant (platelet number/mL of solution) was 10000. The FIGS. 3a and 3b show the conditions of the body area awaiting the skincare stimulation before the treatments and 2 weeks after the treatments, wherein the photographs on the right are the partially magnified pictures of the photographs on the left. The results showed: the body area awaiting the skincare stimulation had wrinkles that either vanished or became less apparent, and spots thereon became lighter in color; showed significantly increased skin elasticity and firmness, and the pores became smaller while the skin became whiter and clearer.

### Embodiment 4

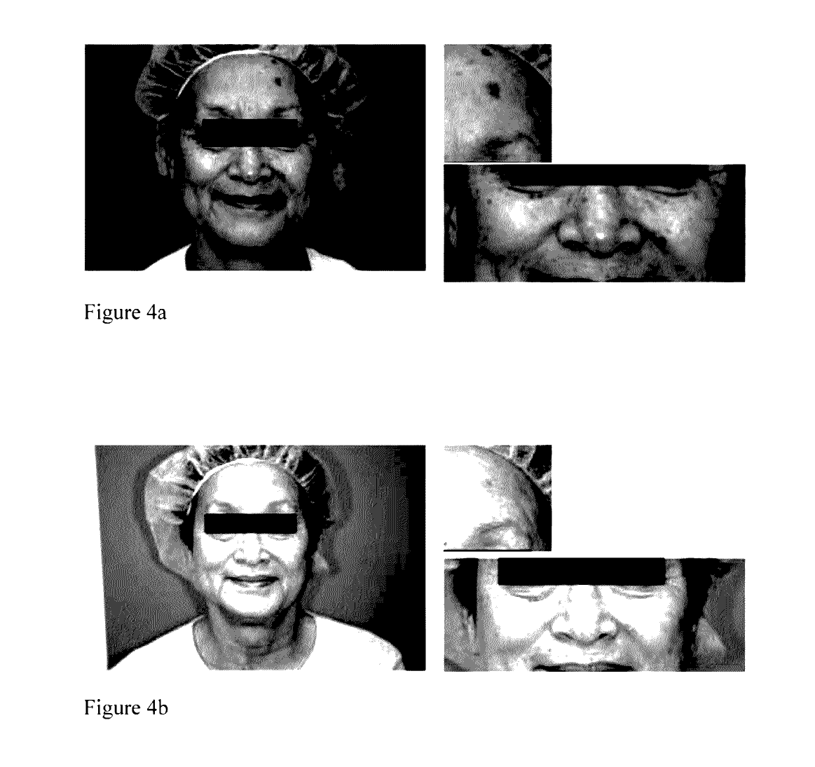

Similar to Embodiment 1, but the platelet dry powder was a homologous platelet dry powder prepared from the blood in a blood bank, and the concentration of the skincare stimulant (platelet number/mL of solution) was 5000. The FIGS. 4a and 4b show the conditions of the body area awaiting the skincare stimulation before the treatments and 4 weeks after the treatments, wherein the photographs on the right are the partially magnified pictures of the photographs on the left. The results showed: the body area awaiting the skincare stimulation had wrinkles that either vanished or became less apparent, and spots thereon became lighter in color; showed significantly increased skin elasticity and firmness, and the pores became smaller and the skin became whiter and clearer.

### Embodiment 5

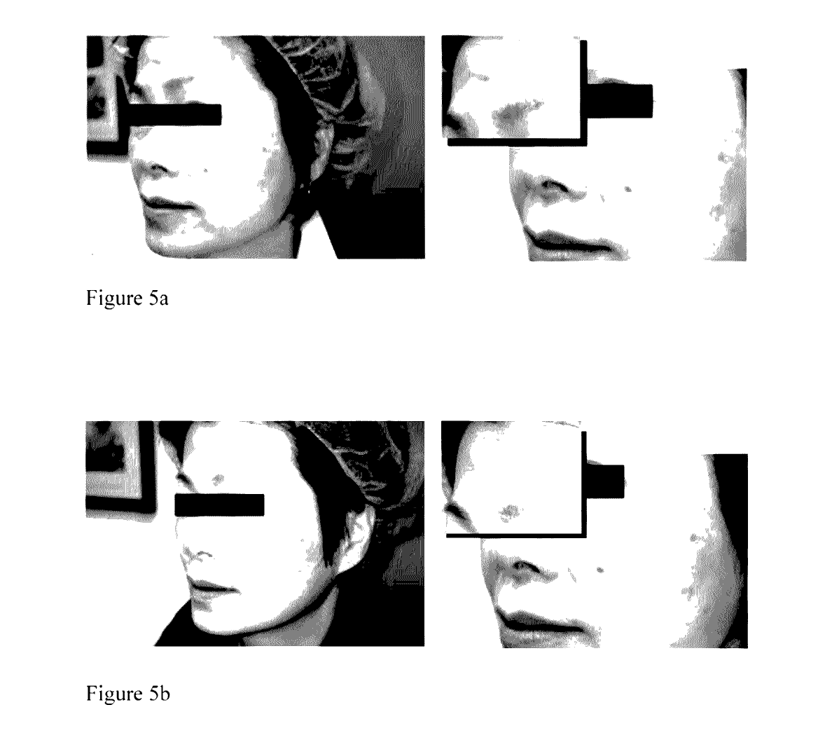

Similar to Embodiment 1, but the platelet dry powder was a homologous platelet dry powder prepared from the blood in a blood bank, and the concentration of the skincare stimulant (platelet number/mL of solution) was 5000. The FIGS. 5a and 5b show the conditions of the body area awaiting the skincare stimulation before the treatments and 4 weeks after the treatments, wherein the photographs on the right are the partially magnified pictures of the photographs on the left. The results showed: the body area awaiting the skincare stimulation had wrinkles that either vanished or became less apparent; scars thereon became lighter (please refer to the picture between FIGS. 5a and 5b); showed significantly increased skin elasticity and firmness; the pores became smaller and the skin became whiter and clearer.

### Embodiment 6

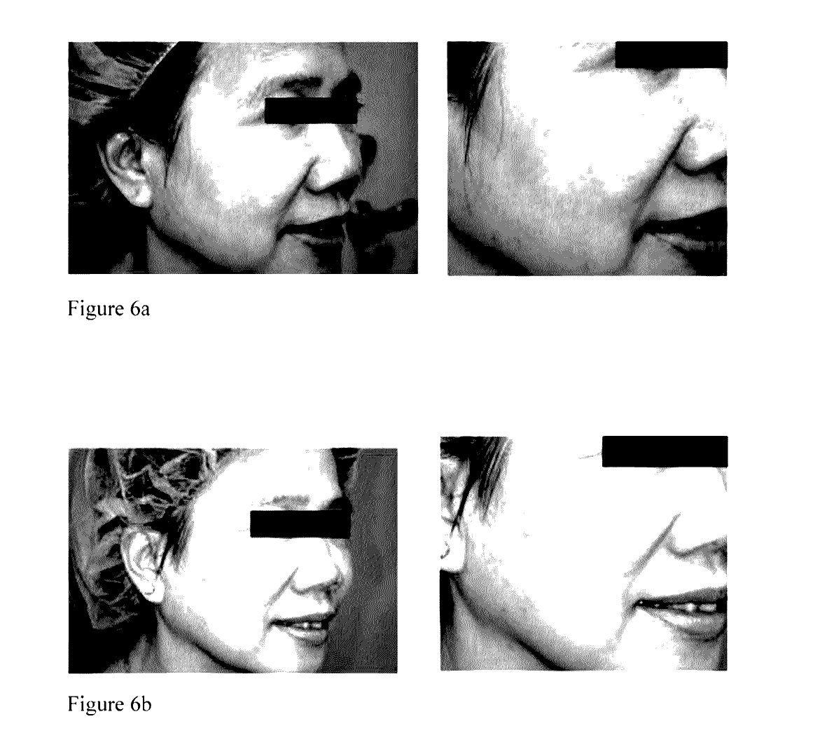

Similar to Embodiment 1, but the platelet dry powder was a homologous platelet dry powder prepared from the blood in a blood bank, and the concentration of the skincare stimulant (platelet number/mL of solution) was 5000. The FIGS. 6a and 6b show the conditions of the body area awaiting the skincare stimulation before the treatments and 7 weeks after the treatments, wherein the photographs on the right are the partially magnified pictures of the photographs on the left. The results showed: the body area awaiting the skincare stimulation had wrinkles that either vanished or became less apparent, and spots thereon became lighter in color; showed significantly increased skin elasticity and firmness, and the pores became smaller and the skin became whiter and clearer.

### Embodiment 7

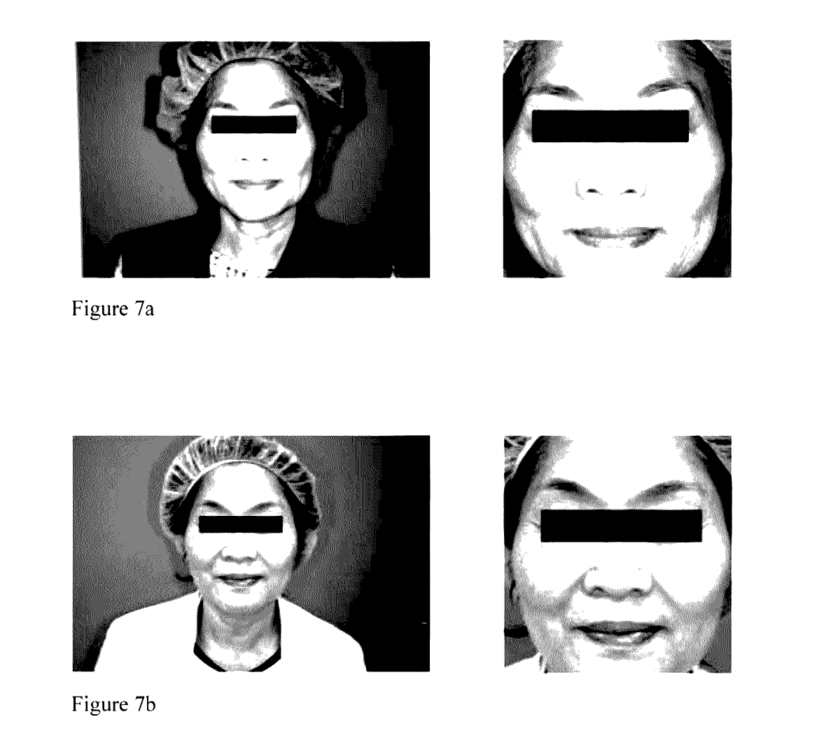

Similar to Embodiment 1, but the platelet dry powder was a homologous platelet dry powder prepared from the blood in a blood bank, and the concentration of the skincare stimulant (platelet number/mL of solution) was 5000. The FIGS. 7a and 7b show the conditions of the body area awaiting the skincare stimulation before the treatments and 7 weeks after the treatments, wherein the photographs on the right are the partially magnified pictures of the photographs on the left. The results showed: the body area awaiting the skincare stimulation had wrinkles that either vanished or became less apparent, and spots thereon became lighter in color; showed significantly increased skin elasticity and firmness, and the pores became smaller and the skin became whiter and clearer.

### Embodiment 8

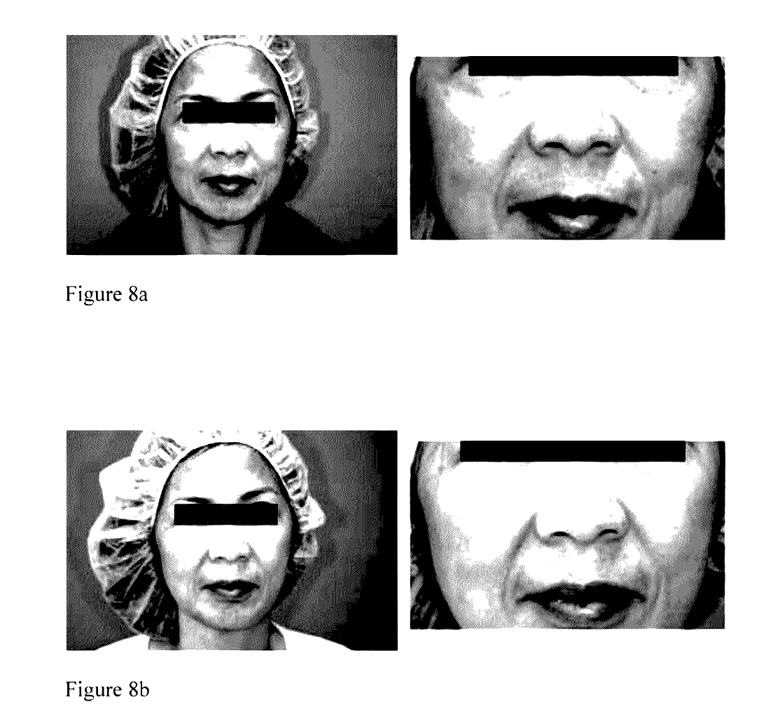

Similar to Embodiment 1, but the platelet dry powder was a homologous platelet dry powder prepared from the blood in a blood bank, and the concentration of the skincare stimulant (platelet number/mL of solution) was 3000. The FIGS. 8a and 8b show the conditions of the body area awaiting the skincare stimulation before the treatments and 24 weeks after the treatments, wherein the photographs on the right are the partially magnified pictures of the photographs on the left. The results showed: the body area awaiting the skincare stimulation had wrinkles that either vanished or became less apparent, and spots thereon became lighter in color; showed significantly increased skin elasticity and firmness, and the pores became smaller while the skin became whiter and clearer.

### Embodiment 9

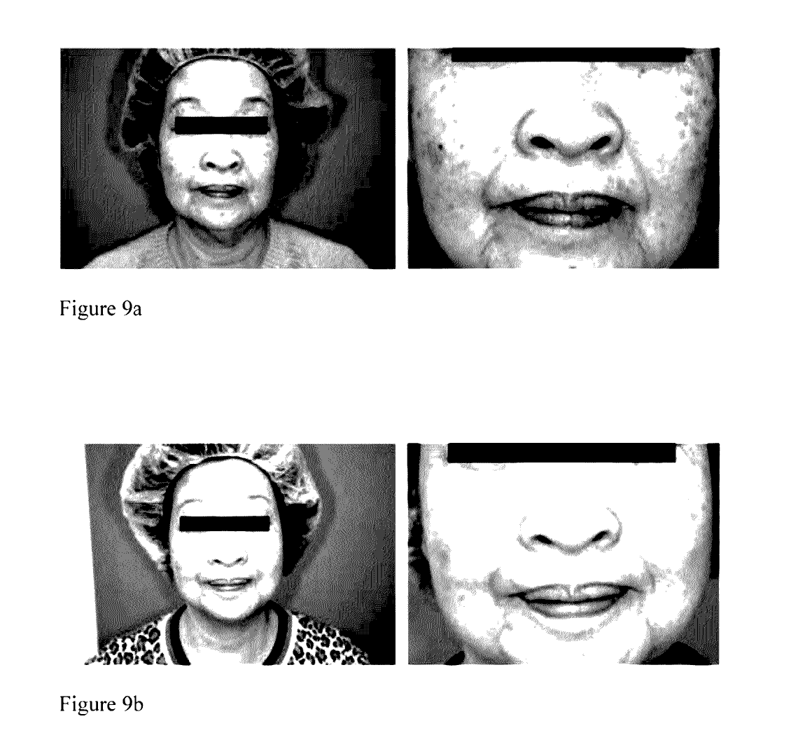

Similar to Embodiment 1, but the platelet dry powder was a homologous platelet dry powder prepared from the blood in a blood bank, and the concentration of the skincare stimulant (platelet number/mL of solution) was 3000. The FIGS. 9a and 9b show the conditions of the body area awaiting the skincare stimulation before the treatments and 21 weeks after the treatments, wherein the photographs on the right are the partially magnified pictures of the photographs on the left. The results showed: the body area awaiting the skincare stimulation had wrinkles that either vanished or became less apparent, and spots thereon became lighter in color; showed significantly increased skin elasticity and firmness, and the pores became smaller while the skin became whiter and clearer.

### Embodiment 10

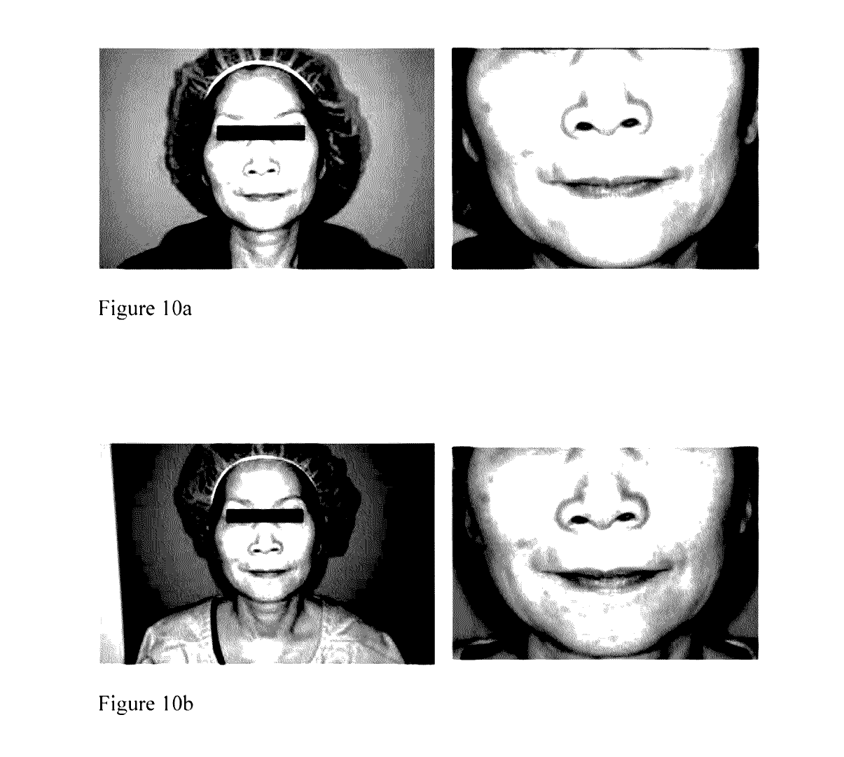

Similar to Embodiment 1, but the platelet dry powder was a heterologous platelet dry powder prepared from bovine blood, and the concentration of the skincare stimulant (platelet number/mL of solution) was 5000. The FIGS. 10a and 10b show the conditions of the body area awaiting the skincare stimulation before the treatments and 3 weeks after the treatments, wherein the photographs on the right are the partially magnified pictures of the photographs on the left. The results showed: the body area awaiting the skincare stimulation had wrinkles that either vanished or became less apparent, and spots thereon became lighter in color; showed significantly increased skin elasticity and firmness, and the pores became smaller and the skin became whiter and clearer.

### Embodiment 11

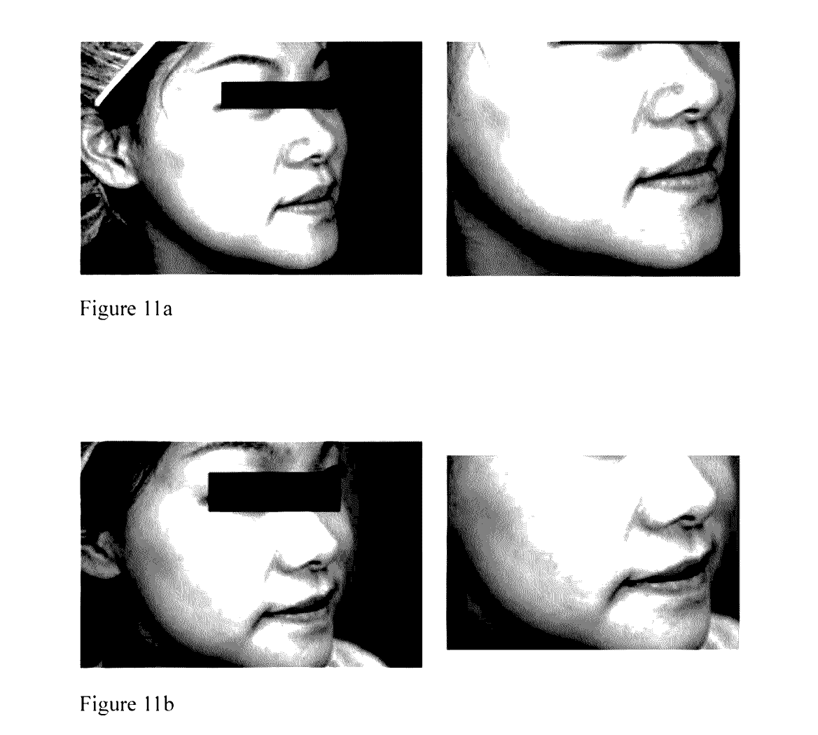

Similar to Embodiment 1, but the platelet dry powder was a heterologous platelet dry powder prepared from bovine blood, and the concentration of the skincare stimulant (platelet number/mL of solution) was 3000. The FIGS. 11a and 11b show the conditions of the body area awaiting the skincare stimulation before the treatments and 8 weeks after the treatments, wherein the photographs on the right are the partially magnified pictures of the photographs on the left. The results showed: the body area awaiting the skincare stimulation had wrinkles that either vanished or became less apparent, and spots thereon became lighter in color; showed significantly increased skin elasticity and firmness; the pores became smaller and the skin became whiter and clearer.

### Embodiment 12

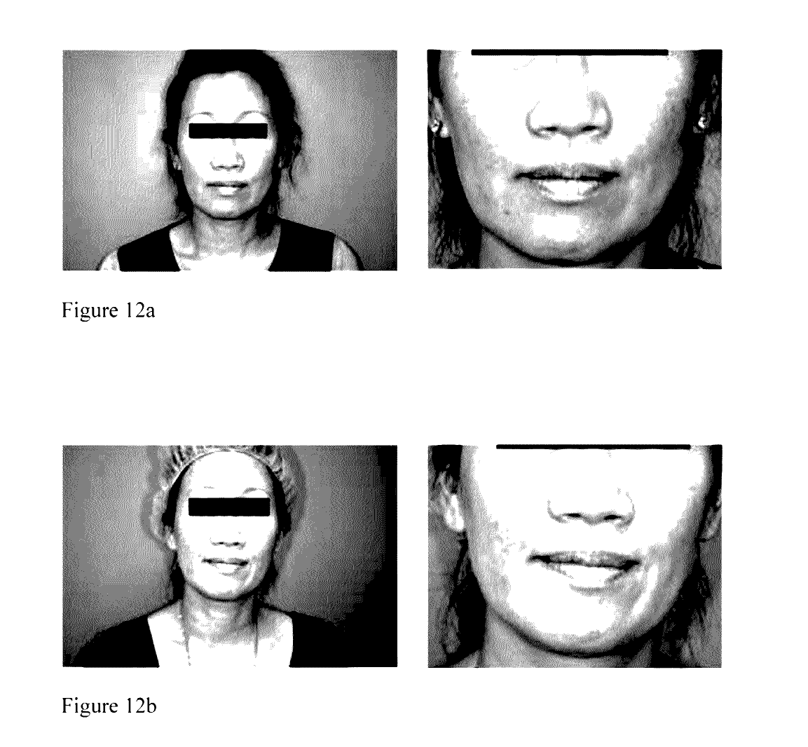

Similar to Embodiment 1, but the platelet dry powder was a homologous platelet dry powder prepared from the blood in a blood bank, and the concentration of the skincare stimulant (platelet number/mL of solution) was 1000. The FIGS. 12a and 12b show the conditions of the body area awaiting the skincare stimulation before the treatments and 19 weeks after the treatments, wherein the photographs on the right re the partially magnified pictures of the photographs on the left. The results showed: the body area awaiting the skincare stimulation had wrinkles that either vanished or became less apparent, and spots thereon became lighter in color; showed significantly increased skin elasticity and firmness; the pores became smaller, and the skin became whiter and clearer.

The results from the embodiments indicate that: with respect to the effects of the treatments, the heterologous PLT appeared to outperform the homologous PLT and the autologous PLT. However, considering the patients' possible concerns, it is preferable to use homologous PLT or autologous PLT, and is more preferable to use autologous PLT.

What is claimed is:

1. A method for treating a skin condition selected from the group consisting of wrinkles, amyloidosis and keratosis pilaris, comprising:
a step for identifying a body area having a condition selected from the group consisting of wrinkles, amyloidosis and keratosis pilaris; and
a step for spraying or spreading a skincare stimulant having platelets over said body area,
wherein the skincare stimulant is a solution having a concentration of 1000-10000 platelets per milliliter of the solution;
wherein a source of the platelets is a platelet dry powder having less than 30% of plasma protein, based on the weight of the platelet dry powder.

2. The method of claim 1 further comprising a step for cleaning the body area before said skincare stimulant being sprayed or spread over the body area.

3. The method of claim 2, wherein said cleaning step comprises rinsing, disinfecting or a combination thereof.

4. The method of claim 3 further comprising an absorption-enhancing step after said cleaning step, and said absorption-enhancing step is executed before, during, or after the spraying or spreading step.

5. The method of claim 4, wherein said absorption-enhancing step is executed before the spraying or spreading step by utilizing a laser treatment, micro-needle guiding, or physical stimulation.

6. The method of claim 1, wherein the solution uses water or saline as a solvent thereof.

7. The method of claim 1, wherein the platelets are autologous platelets.

8. The method of claim 1, wherein the concentration is 3000 platelets per milliliter of the solution.

9. The method of claim 8, wherein the concentration is 5000 platelets per milligram of the solution.

10. The method of claim 1, wherein said skin condition is wrinkles.

11. The method of claim 1, wherein said skin condition is amyloidosis.

12. The method of claim 1, wherein said skin condition is keratosis pilaris.

13. A method for treating a skin condition selected from the group consisting of wrinkles, amyloidosis and keratosis pilaris, comprising:
a step for identifying a body area having a condition selected from the group consisting of wrinkles, amyloidosis and keratosis pilaris;
a step for preparing a skincare stimulant having platelets comprising mixing water or saline with a platelet dry powder to form a solution having a concentration of 1000-10000 platelets per milliliter of the solution, wherein the platelet dry powder contains less than 30% of plasma protein, based on the weight of the platelet dry powder; and
a step for spraying or spreading the skincare stimulant over said body area.

14. A method for treating a skin condition selected from the group consisting of wrinkles, amyloidosis and keratosis pilaris, consisting essentially of:
a step for identifying a body area having a condition selected from the group consisting of wrinkles, amyloidosis and keratosis pilaris; and
a step for spraying or spreading a skincare stimulant having platelets over said body area,
wherein the skincare stimulant is a solution having a concentration of 1000-10000 platelets per milliliter of the solution;
wherein a source of the platelets is a platelet dry powder having less than 30% of plasma protein, based on the weight of the platelet dry powder.
